(19) United States
(12) Patent Application Publication
(10) Pub. No.: US 2008/0104737 A1
(43) Pub. Date: May 8, 2008

(54) Protective Article

(75) Inventor: Stephen H. Shepherd, West Palm Beach, FL (US)

Correspondence Address: AKERMAN SENTERFITT, P.O. BOX 3188, WEST PALM BEACH, FL, 33402-3188, US

(73) Assignee: Ringstar, Inc., West Palm Beach, FL (US)

(21) Appl. No.: 11/557,667

(22) Filed: Nov. 8, 2006

(51) Int. Cl. A41D 13/00; A41D 19/00; A41D 13/08; A61F 5/00

(52) U.S. Cl. 2/20; 2/161.1; 2/16; 602/21

(57) ABSTRACT

A protective article is provided having a body with a finger portion and a forearm portion. The body extends from the first finger knuckles past a wearers wrist joint. The protective article has a padding extending over at least a portion of the body, with a knuckle pad region and a forearm pad region that extends from beyond the first finger knuckles past a wearer's wrist joint. The protective article can have a flexible joint at the juncture between the knuckle and the forearm pad regions and a rigid forearm guard covering a portion of the forearm pad region, extending from the flexible joint past a wearer's wrist joint. A wrist wrap can be provided that has at least one fastener attached to the body, operable to secure the forearm guard adjacent to a dorsal side of a wearer's wrist to substantially secure a wearer's wrist joint.

### CROSS REFERENCE TO RELATED APPLICATIONS

(Not Applicable)

### STATEMENT REGARDING FEDERALLY SPONSORED RESEARCH OR DEVELOPMENT

(Not Applicable)

### BACKGROUND OF INVENTION

1. Field of the Invention

The present invention relates to a wearable protective article, and more particularly to a protective article that can be used during physical activities such as contact sports.

2. Description of the Related Art

Hand and wrist protection has always been needed for activities that place prolonged stress on the hands, wrists, and forearms. For example, hands, wrists, and forearms of construction workers are subject to stress due to lifting and manipulation of heavy construction materials or prolonged use of power tools. Similarly, delivery workers are also prone to similar stresses due to the constant lifting and manipulation of packages. Athletes also face similar problems. For example, in martial arts and boxing, the hands, fingers, and knuckles are constantly under stress with each punch that is thrown.

Without proper hand padding protection during such activities, hands can become damaged with such injuries as a complete break, fractures, ligament damage. Additionally, because the extreme force used by such athletes radiates down the wrist and arms, the wrists and forearms can also be damaged with injuries such a complete break or fracture. All of these injuries can result in swelling of the hands and forearms. Because of these situations a hand wrap is generally used to support hands, wrists, and forearms, to provide some protection to these areas.

Laborers generally use heavy gloves in order to protect their hands, using any number of devices, such as straps, to secure the protective gloves on their hands. Participants in boxing and martial arts go to extreme means in order to protect their hands and wrists from breaks and fractures. For example, one hundred seventy (170) inches of hand wrap, which constitutes fourteen (14) feet, is used by golden gloves and amateur boxers. Professional boxers and trainers can spend 45 minutes to 1 hour wrapping a fighter's hands and knuckles with gauze. A similar situation is faced by martial artists, such as kick boxers and mixed martial arts (MMA) competitors. Furthermore, the concern over preventing such injuries is so great that the governing bodies for such sports not only require standardized hand wrap use, but also inspection prior to competition

Hand wrapping is a generally recognized means of providing protection to the hands, wrists, and forearms. With more emphasis in the modern world on maintaining good health and physical condition, more individuals are faced with the task of applying a hand wrap to their wrists and often do so incorrectly.

For example, in martial arts, proper hand wrapping technique is necessary because hand wraps are used not only as a protective device during training with punching bags, punching targets, or similar training devices, but also as additional protection under boxing, kickboxing, and other similar types of fighting gloves. As such, improper hand wrapping can result in the inability to properly and comfortably insert hands into a fighting glove. In addition, another result of improper hand wrap technique can be injuries because the hand wrap was not properly secured to adequately support the wrist. Additionally, such hand wraps are increasingly utilized by children engaging in martial arts, making the likelihood of improper wrapping, and therefore injury, higher due to the wearers inexperience. Another result is that a student can learn improper techniques during instruction, because of improper alignment of hand, wrist and forearm, which can increase the likelihood of wrist injuries in particular.

Traditional hand wrapping, while providing support to the hands, wrists, and forearms, has several drawbacks. First, hand wrapping can be time consuming. As stated above, professional boxers can spend up to an hour wrapping their hands, however, the average person typically will not wish to invest so much time to hand wrapping. This is especially apparent in the case of athletes, who often spar or train for short periods as part of their exercise regime and continued usage of a traditional hand wrap is not necessary. In the case of laborers or other users, the most careful hand wrapping may not be necessary, but at the very least, consistent hand wrapping should be used so that at least minimal support is provided.

Second, hand wraps may not be reusable. For example, the some wearers will use a roll of gauze or other wrapping material and dispose of the gauze after the end of the activity. Reusing gauze is not always practical since during use, wrapping, and unwrapping of the gauze hand wrap, the gauze tends to become damaged and unsanitary. Rolls of elastic bandages may reusable, but are thicker and bulkier than gauze. Therefore, in order to provide sufficient support to the hands and wrist, a large length of elastic bandage is necessary. In cases where a hand wrap under a glove is desired, a large bulky hand wrap would make it difficult to slip on a glove.

Devices in the art capable of providing support and protection to the hands, wrist, and forearm are generally limited to bulky training or target devices used for boxing or kickboxing training, but such devices cannot be used in concert with other gloves, due to their large size. Furthermore, such devices limit or impair use of the hands and fingers, which can be important in construction work or in certain martial arts.

Smaller protective articles in the art generally comprise a small pliable glove type device utilizing one or more elastic hand wraps or straps, but such devices still allow an unacceptable amount of hand and wrist movement due to the lightweight materials used their in construction.

Therefore, what is needed is a protective article that is simple to use by children and adults alike. Such a protective article can provide adequate protection and stability for the hand, wrist, and forearm, while maintaining proper hand, wrist, and forearm alignment for wearers. The protective article can be easy to put on and remove, and can be resilient enough so that it is reusable. The protective article can also be small and lightweight, allowing the wearer to secure his hand and wrist with little or no wrist movement. The protective article can also allow the wearer to use the article alone or under a glove.

### SUMMARY OF THE INVENTION

The present invention is directed to a protective article that provides stability and protection for the fingers, hands, wrists, and forearms of the wearer. The protective article can be small, light, and easy to put on and remove. The protective article can be used as a training device by itself, or in concert with a fighting glove, such as a boxing or kickboxing glove.

In a first arrangement of the invention, a protective article is provided comprising a body having a finger portion and a forearm portion, wherein the forearm portion extends from the first finger knuckles to a location past a wearer's wrist joint; a padding extending over at least a portion of the dorsal side of the body, having a knuckle pad region and a forearm pad region, wherein the forearm pad region extends from beyond the first finger knuckles to a location past a wearers wrist joint; a flexible joint at the juncture between said knuckle pad region and said forearm pad region; a substantially rigid forearm guard covering at least a portion of the forearm pad region, wherein the guard extends from the flexible joint to a location past a wearer's wrist joint; and, a wrist wrap attached to the body, wherein the wrist wrap is designed to secure the forearm guard adjacent the dorsal side of a wearer's wrist and secure a wearer's wrist joint, and includes one or more fasteners operable to fasten the wrist wrap in place.

In some arrangements of the invention, the knuckle pad region and said forearm pad region are separate pieces of padding. In other arrangements of the invention, the thickness of the knuckle pad region can be greater than the thickness of the forearm pad region.

In some arrangements of the invention, the forearm guard and the forearm pad can be adapted to generally match the dorsal contour of a wearer's hand, wrist and forearm. In other arrangements of the invention, the forearm guard can comprises a plurality of adjacent forearm guard strips, where each one of the strips can extend from the flexible joint to a location past a wearer's wrist joint.

In some arrangements of the invention the wrist wrap can further comprise an long elastic material. In other arrangements of the invention, the wrist wrap can extend from a lateral edge located proximally to the wearer's thumb.

In some arrangements of the invention, the pliable glove can further comprise a fingerless glove, where the finger portion and the thumb portion extend only a part way along a wearer's fingers and thumb. In other arrangements of the invention, the pliable body can have a closed finger portion, an open palm portion and a closed wrist portion. In some arrangements, this wrist portion can comprises an elastic band attached at each lateral edge of the body.

In another arrangement a protective article can be provided comprising a body having a finger portion and a forearm portion; a forearm guard being substantially rigid and extending from at least said forearm portion to a location past a wearer's wrist joint; and a wrist wrap having at least one fastener and operable to secure the forearm guard adjacent to a dorsal side of the wearers wrist joint to support the wearer's wrist joint. The forearm portion can extend from a wearer's first finger knuckles to a location past the wearer's wrist joint. The protective article can further comprise a padding extending over at least a portion of a dorsal side of the body, with the padding having a knuckle pad region and a forearm pad region, wherein the forearm pad region extends from beyond the first finger knuckles to a location past the wearer's wrist joint; and a flexible joint at the juncture between the knuckle pad region and the forearm pad region, wherein the forearm guard covers at least a portion of the forearm pad region.

In another arrangement a protective article may be provided comprising: a body having a finger portion and a forearm portion, wherein the forearm portion extends from a wearer's first finger knuckles to a location past the wearer's wrist joint; a padding extending over at least a portion of a dorsal side of the body, with the padding having a knuckle pad region and a forearm pad region, wherein the forearm pad region extends from beyond the first finger knuckles to a location past the wearer's wrist joint; a casing surrounding at least a portion of the padding; a flexible joint at the juncture between the knuckle pad region and the forearm pad region, wherein the forearm guard covers at least a portion of the forearm pad region; a forearm guard being substantially rigid and extending from at least the flexible joint to a location past a wearers wrist joint; and a wrist wrap having at least one fastener and operable to secure the forearm guard adjacent to a dorsal side of the wearer's wrist joint to support the wearer's wrist joint.

In another arrangement, a method of supporting a wearers wrist joint is provided comprising: positioning a substantially rigid forearm guard on a dorsal side of a wearers hand from at least in proximity to a wearers knuckles to a location past a wearers wrist joint; holding the substantially rigid forearm guard in position via a glove body; and securing the substantially rigid forearm guard against the dorsal side of the wearers hand via a wrist wrap to support the wearer's wrist joint.

The forearm guard may extend from at least the flexible joint to the location past a wearer's wrist joint. The wrist wrap can be attached to the body at a first end and have the at least one fastener at a second end. The knuckle pad region and the forearm pad region may be separate pieces of padding. The thickness of the knuckle pad region can be greater than the thickness of the forearm pad region. The forearm guard and the forearm pad region may be shaped to generally conform to a dorsal contour of a wearer's hand, wrist or forearm. The wrist wrap can comprise an elastic material.

The wrist wrap may be attached to the body at a lateral edge of the body located proximally to a wearer's thumb. The body may comprise a fingerless glove, with the finger portion extending only a part way along a wearers fingers. The palmar side of the body can comprise a closed finger portion, an open palm portion and a closed wrist portion. The wrist portion may comprise an elastic band attached at each lateral edge of the body. The forearm guard can comprise a plurality of adjacent forearm guard strips, with each one of the strips extending from the flexible joint to a location past the wearers wrist joint.

The method may further comprise positioning padding on a dorsal side of a wearers hand; and holding the padding in position via a casing surrounding the padding and operably connected to the glove body, wherein the glove body has a finger portion and a forearm portion, and wherein the forearm portion extends from a wearers first finger knuckles to a location past the wearers wrist joint. The method can further comprise at least one of the substantially rigid forearm guard or the padding being removable from the glove body.

### DESCRIPTION OF THE DRAWINGS

There are shown in the drawings, arrangements that are presently discussed; it being understood, however, that the invention is not limited to the precise arrangements and instrumentalities shown.

FIG. 1 is a dorsal view of a device in accordance with an arrangement of the invention.

FIG. 2 is a palmar view of a device in accordance with an arrangement of the invention.

FIG. 3a is dorsal view of the padding arrangement in accordance with an arrangement of the invention.

FIG. 3b is a lateral view of the padding arrangement in accordance with an arrangement of the invention.

FIG. 3c is a cross-sectional view of the padding arrangement in accordance with an arrangement of the invention thru the line 3c-3c in FIG. 3a.

### DETAILED DESCRIPTION OF THE DRAWINGS

With reference now to the various figures in which like elements are identically numbered throughout, a description of the various arrangements of the present invention will now be provided. While the invention is disclosed in the context of a single arrangement, it will be appreciated that the invention can include numerous modifications from the preferred arrangement.

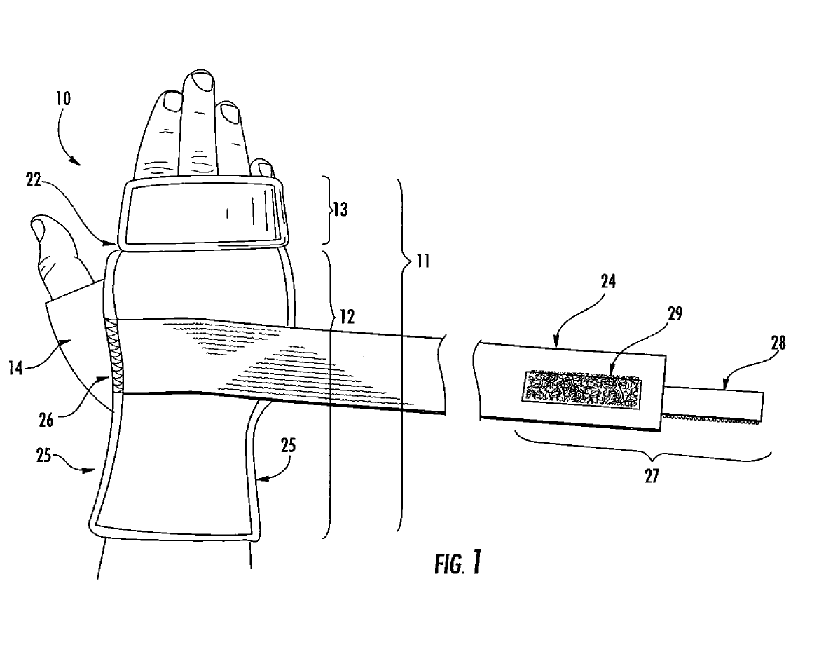

With reference to FIGS. 1-3, a protective article is shown at reference numeral 10. It should be understood that the protective article 10 can be used for a variety of activities in which support of one or more of the hands, wrist, and forearm is necessary. In particular, article 10 can be used for activities when it is desired to immobilize or substantially impede movement of the wrist joint in order to protect the wearer from harm. Such activities may include construction work, delivery work, or athletic activities that place the wrist joint at risk for harm. For example, athletic activities, including martial arts sports such as boxing, karate, kickboxing, tae kwon do, and kung-fu, can place great stress on the hands and wrists during sparring or even training. One application of the protective article 10 is as a hand wrap for martial arts sports.

While the invention is described with respect to its use in martial arts, it should be appreciated that the device has application in any field where wrist support is required. The features of the illustrated arrangement of the protective article device 10 are particularly well adapted for martial arts, as protective article 10 provides protection from impacts and provides stability for the hand and wrist during training. However, article 10 is suited for other activities, including both contact and non-contact activities, where support for the wrist is sought.

The protective article 10 comprises a body 11 constructed from one or more pliable materials such as leather, vinyl, or cloth. The material of body 11 can be sufficiently resistant to impact so that it can be used for sparring or punching bag training. In addition, because the skin will encounter moisture, primarily from sweat, the material of body 11 can comprise a material that can resist damage caused by contact with moisture. In some arrangements, a vinyl backed cloth can be used for some or all of the body 11. Additionally, in other arrangements, the body 11 may be reinforced to protect stress areas from tearing apart or otherwise being damaged after repeated use or stress.

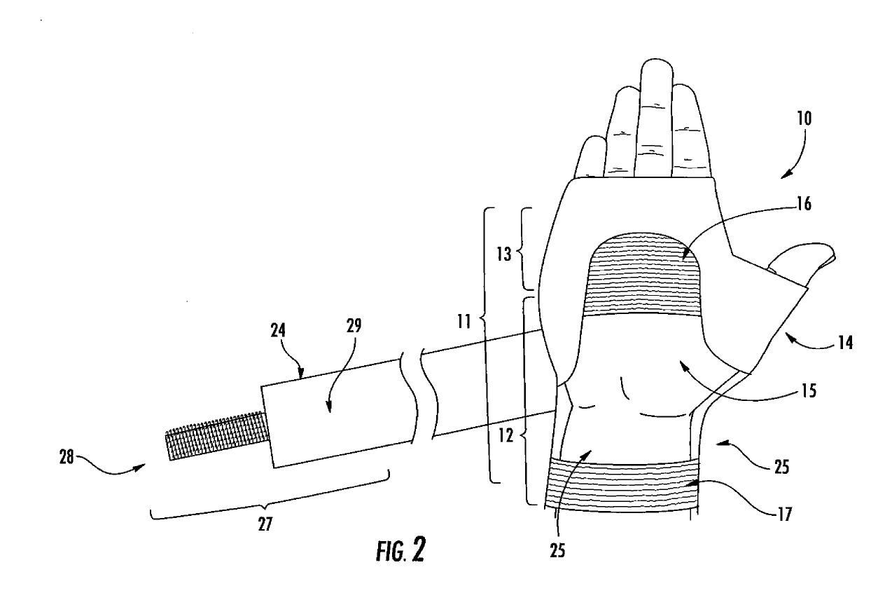

The body 11 comprises two general regions, as shown in FIGS. 1 and 2. The first region can be referred to as the forearm portion 12, which provides protection for one or more of the hand, wrist, and forearm, and the second region can be referred to as the finger portion 13, which provides protection to the fingers. In some arrangements, a third region, a thumb portion 14, can also be provided. In the various arrangements, the forearm portion 12 is adapted to extend from beyond the first finger knuckles to a location past a wearer's wrist joint, as shown in FIG. 2.

In some arrangements, the body 11 may be configured to completely cover the hands, wrists, and forearms of the wearer. However, in other arrangements, such as the illustrated arrangement in FIG. 2, the body 11 can be configured to only cover a portion of the hand, wrist, or forearm of the wearer. In such arrangements, complete coverage of the palmar side of the wearers limb may not be necessary where only the dorsal side of the wearers limb is prone to injury.

As shown in the illustrated arrangement in FIG. 2, the palmar side of body 11 may be further comprised of an open palm region 15. In such arrangements, the palmar side of the body 11 may only comprise of an upper hand portion 16 and a wrist portion 17, leaving the palm of the wearer completely exposed. In the illustrated arrangement as shown in FIG. 2, the wrist portion 17 comprises an elastic band, as does a portion of the upper hand portion 16. The advantage of such a configuration is that the body may be securely placed on the wearers hand and kept in place by the elastic in the wrist portion 17 and the elastic in the upper hand portion 16.

However, in other arrangements, elastic may not be used at all in either the wrist portion 17 or the upper hand portion 16. In such arrangements, a strap using some type of complementary fastener arrangement may be used for the wrist portion. Such fastener arrangements may include buttons, hook and loop fasteners, or buckles. In other arrangements, the upper hand portion 16 can comprise individually constructed finger loops. In such arrangements, the finger loops can comprise simple loops constructed of the same materials as the pliable body 11. In other arrangements, the finger loops can comprise individual elastic portions.

In some arrangements, the body 11 may be configured to completely cover the fingers and thumb of the wearer. However, as shown in the illustrated arrangement in FIGS. 1-3, the body 11 may also comprise a fingerless body 11. In such arrangements, the finger portion 13 and any thumb portion 14 are adapted to extend only a part way along the wearers fingers and thumb. Such a configuration is advantageous in that finger dexterity and tactile ability is not affected by the protective article 10, allowing the wearer to use his fingers normally in spite of wearing the protective article.

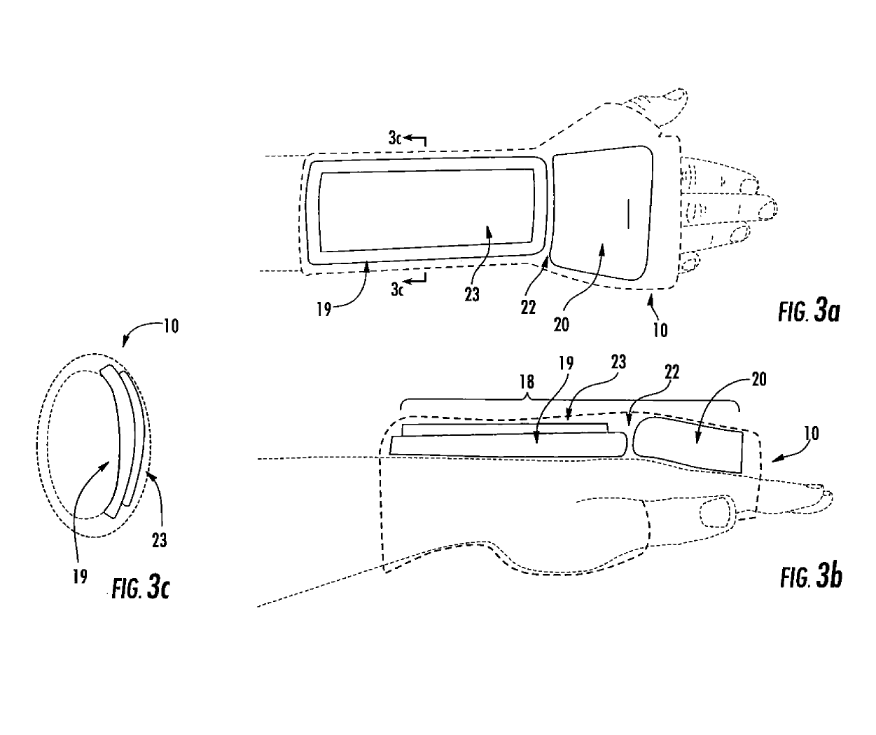

In the various arrangements, a padding 18 can be provided to further protect the wearer's fingers, hands, wrists, and forearm, as shown in FIGS. 3a-3c. The padding 18 can be configured to extend over at least a portion of the dorsal side of the body 11. In some arrangements, the padding 18 can be disposed on the exterior of the body 11. In other arrangements, the glove padding 18 may be disposed on the interior of the body 11, but still extend over the dorsal side of the body 11.

The padding 18 can be located throughout the body 11 or only in certain areas of the body. In the illustrated arrangement, the padding 18 is on those areas of the body 11 which cover portions of the fingers, hand, and wrist that are more likely to be injured during athletic activities involving kicking, such as kick boxing and martial arts. However, the present disclosure contemplates the positioning of padding 18 on other portions of body 11 and such portions may be chosen based upon the particular activity for which the article 10 will be used. The present disclosure contemplates the padding 18 having any suitable thickness, and may have a thickness of between 0.25 and 0.5 inches, such as 0.375 inch padding. The particular thickness can be chosen based upon the particular event for which the article 10 will be used. In the various arrangements, open cell foam, closed cell foam or any combination thereof may be used for the padding 18. Open cell foam padding is typically softer and can be used for increased comfort, but the closed-cell foam padding can typically offer more injury protection. Areas of the body 11 which are not thickly padded, such as the palmar side of the body 11 may be padded with any suitable thickness of padding, such as 0.125 inch, or may be replaced by open portions or mesh to allow for added ventilation of the hand of the wearer. The present disclosure also contemplates other types of material being utilized for padding 18. Such other materials may be chosen based upon the particular activity for which the article 10 will be used as well as other factors including cost and facilitating manufacture.

The padding 18 material preferably has a high degree of resiliency and excellent shock absorption properties In some arrangements, the padding 18 material can be a closed-cell chemically cross-linked polyethylene or polyolefin foam, such as the Minicel® products manufactured by the Voltek Division of the Sekisui America Corporation. The closed-cell foams produced under the Minicel trademark include the L200, L300, L200F, L380, LS200, LS300, LS380, M200, M300, M380, MS200, MS300, MS380, T200, T300, TS200, TS300 and TS380 foams, which have excellent strength and shock absorption properties. In addition, these foams have a low degree of water absorption. Although not limited in this regard, closed-cell foam padding suitable for use as padding 18 preferably has a durometer of around 0.253. The foams can also preferably have a density of between approximately 1.5 and 4.5 pcf, a compression strength of between approximately 3 and 23 psi at approximately 25% deflection and between approximately 9 and 42 psi at approximately 50% deflection, and a tensile strength of between 28 and 145 psi. However, other ranges of density, compression strength and tensile strength for padding 18 are contemplated by the present disclosure.

The padding 18 can comprise two general regions, as shown in FIGS. 3a and 3b. The first region can be referred to as the forearm pad portion 19, to provide protection of the hand, wrist, and forearm, and the second region can be referred to as the knuckle pad portion 20, to provide protection to the fingers. In the various arrangements, the forearm pad portion 19 is adapted to extend from the first finger knuckles to a location past a wearer's wrist joint, as shown in FIG. 3b. In some arrangements, the padding 18 can comprise a single continuous piece of padding. The padding 18 can be of uniform or generally uniform thickness or can be of varying thickness. In other arrangements, as shown in FIGS. 3a and 3b, the forearm pad portion 19 and the knuckle pad portion 20 may comprise separate pieces of padding 18. The present disclosure also contemplates the use of two or more separate pieces of padding 18 that are connected or fastened together. In such exemplary embodiments, the particular fastening structures between the two or more separate pieces of padding 18 can be chosen to provide adequate protection and comfort to the wearer. The present disclosure contemplates various sizes and shapes of the two or more pieces of padding 18 being used along the dorsal side of article 10.

In some arrangements, as shown in FIG. 3b, the knuckle pad portion 20 may be configured to be thicker than the forearm pad portion 19. The advantage of such an arrangement being that when used in fighting sports, the wearer is more likely to hit an opponent or punching bag with his closed fist, and the increased padding reduces likelihood of injury to his fingers or knuckles.

In the various arrangements, the padding 18 can be protected from damage, by impact or by moisture, by using a protective casing or sheath. In some arrangements, the casing may be separate from the body 11 and attached to the body 11 later. In other arrangements, the casing may be defined by using multiple layers of material to construct the body 11 and defining a region in between some of the layers as the casing or sheath. In yet other arrangements, the casing may comprise an attached pad covering for the padding 18 disposed on the body 11. In the various arrangements, the casing may comprise pliable materials, similar to the body 11, to match the body 11, or as necessary due to the location of the padding 18. While in the exemplary embodiment of article 10, the casing covers all of the padding 18, the present disclosure contemplates the casing covering only a portion of the padding.

In the various arrangements, the protective article 10 is also comprised of a flexible joint 22 at the juncture between the knuckle pad region 20 and the forearm pad region 19. In some arrangements, a flexible joint 22 may be naturally formed when the padding 18 comprises two separate padding regions, as discussed above. In other arrangements, a stitched seam through the padding 18 may be used to define the flexible joint 22, which can act as a hinge without the need to provide two separate padding regions. The present disclosure contemplates other structures and techniques being used to provide a hinge along flexible joint 22 including decreasing the thickness of a continuous padding 18 along the flexible joint region. The amount of the decreased thickness of the padding along the region of flexible joint 22 can be chosen based upon various factors including the amount of mobility that the flexible joint is to allow.

In the various arrangements, the protective article 10 may also comprise a forearm guard 23. By way of example, not by way of limitation, the forearm guard 23, in the various arrangements, can be adapted to extend over at least over a portion of the hand to a location past the wearer's wrist. In the illustrated arrangement in FIGS. 1-3, the forearm guard 23 is adapted to extend from the first finger knuckles to a location just past the wearers wrist. The forearm guard 23 can be constructed from any substantially rigid material, such as plastic, metal, fiberglass, or ceramic. In some arrangements, the forearm guard 23 can comprise a plurality of forearm guard strips, oriented along the wearers arm to more easily follow the contour of the wearers arm. However, in the illustrated arrangement, the forearm guard 23 is constructed from a single piece. Such an arrangement provides for simpler construction and greater stability provided to the hand, wrist, and forearm of the wearer.

In the illustrated arrangement, a thin, lightweight plastic can be used to provide stability and reduce the weight of the protective article 10. In such arrangements, a plastic forearm guard 23 is also preferred since other materials tend to shatter, break, or deform under stress, resulting in sharp edges that can injure the wearer or an opponent.

In some arrangements, the shape of the forearm guard 23 may be adapted to conform to the general shape of the hand, wrist, or forearm, as shown in FIG. 3c. In the illustrated embodiment, the curvature of the forearm guard 23 is adapted to match the natural curvature of a wearers arm. In the various arrangements, as shown in FIGS. 3a, 3b, and 3c, the forearm guard 23 is disposed on the forearm pad portion 19, such that the forearm pad portion 19 is located between the forearm guard 23 and the wearer, so that the forearm guard 23 does not rub or press directly upon the wearer, such that the protective article 10 remains comfortable. Additionally, when the wearer is struck on the forearm or dorsal side of the wrist, the forearm pad portion 19 provides additional protection to the wearer.

In some arrangements, the forearm guard 23 and the forearm pad portion 19 can be attached together by adhesive. In other arrangements, the padding 18 can constructed with a forearm pad portion 19 having a cutout region in which the forearm guard 23 can be inserted. In other arrangements, the forearm guard 23 may be encased in the forearm pad portion 19. In such embodiments, methods including, but not limited to, injection molding or continuous foam extrusion may be used to encase the forearm guard 23. The present disclosure also contemplates the use of various sizes and shapes for the forearm guard 23. For example, the length of the forearm guard 23 along the dorsal side of the wearers wrist or forearm can be increased or decreased by the use of a particular forearm guard, thus adapting the article for the needs of the particular wearer.

In the various arrangements, the protective article 10 may also comprise at least one wrist wrap 24 configured to secure the forearm guard 23 in place adjacent to the dorsal side of a wearers wrist, thus securing the wrist in place. In the illustrated arrangement, the wrist wrap 24 comprises a thin single strip of material attached to the body 11.

In some arrangements, the wrist wrap 24 may extend from a portion of one of the lateral edges 25 of the body. In the illustrated arrangement, the wrist wrap 24 extends from an attachment point 26, proximal to the thumb portion 14. The wrist wrap 24 may also employ one or more fasteners 27 to fasten the forearm guard 23 in place. In the illustrated arrangement, a complementary set of fasteners 27 are disposed on a first fastener portion 28 attached to the free end of the wrist wrap 24 and on a second fastener portion 29 on the wrist wrap 24 itself. In the illustrated arrangement, the complementary fasteners 27 comprise hook and loop fasteners. In other arrangements, other types of fasteners may be used, however, hook and loop fasteners, by their construction, can reduce the risk of injury to the wearer and opponents due to a lack of sharp edges or hard materials. In the various embodiments, the wrist wrap 24 is not limited to the illustrated arrangement. In some arrangements, the wrist wrap 24 can comprise a wide elastic strip of material designed to wrap only once around the wrist. In other embodiments, the wrist wrap 24 can also comprise multiple strips of materials to further secure the forearm guard.

In operation, as shown in the illustrated arrangement in FIGS. 1-3, the wearer inserts his hand into the protective article 10. The elastic comprising the wrist portion 17 and the elastic comprising the upper hand portion 16 on the palmar side of the pliable body 11 initially secure the protective article 10 on the wearer's hand. Additionally, the protective article 10 is further secured in place by properly adjusting the position of the contoured forearm guard 23 so that the forearm guard 23 properly covers the dorsal side of the wearers hand, wrist, and/or forearm. Once the protective article 10 is in place on the wearers hand, the wearer can then wrap the wrist wrap 24 tightly around the wearers hand, wrist, and the forearm guard 23. The wrist wrap 24 and the forearm guard 23 secure the wrist joint, allowing minimal, if any, movement of the wrist, providing added stability to the wearer's wrist and ensuring proper alignment of the hand, wrist, and forearm. In arrangements where an elastic wrist wrap 24 is used, the wrist wrap 24 can be wrapped even tighter, allowing for a more secure fit and increased stability of the wrist joint. Similarly, a contoured forearm guard 23, as shown in FIGS. 1-3, also allows the wrist wrap 24 to be wrapped tighter, allowing for a more secure fit and increased stability of the wrist joint. Once the entire length of wrist wrap 24 is wrapped around the wearers wrist, the first fastener strip 28 can be attached to the to the second fastener strip 29, so that the set of complementary fasteners 27 can hold the wrist wrap in place. The wearer can then repeat the process on a second hand with a second protective article 10, if necessary. For example, the wearer can then proceed with his training or insert his wrapped hands into fighting gloves. Once the wearer no long needs to use the protective article 10, the process can be reversed to remove the protective article 10.

It will be appreciated that in the various embodiments, the wrist wrap 24 could be separated, intentionally or inadvertently, from the body 11. For example, the wrist wrap 24 could tear at the attachment point 26. In some embodiments, the protective article 10 could comprise of an additional fastening loop also attached at the attachment point 26. In these arrangements, when the wrist wrap 24 is torn off at the attachment point, the torn end of the wrist wrap 24 can be attached to or tied to the fastening loop in order continue use of the protective article 10. However, in other embodiments, the additional fastening loop may be used as the normal method to attach the wrist wrap 25 to the protective article. In other embodiments, the a complementary set of fasteners may be disposed on the body 11 and a second end of the wrist wrap 24. In these arrangement, the complementary fasteners many be used to attach the wrist wrap to the body 11, prior to wrapping the wrist wrap 24 around the wearer's hand and wrist. In such arrangements, many types of fasteners may be used, but hook and loop fasteners, by their construction, can reduce the risk of injury to the wearer and opponents due to a lack of sharp edges or hard materials.

It will be appreciated that the size of the protective article 10 can be altered to accommodate the different sizes associated with use by men, women, and children. It is expected that the size of the forearm pad portion 19 and forearm guard 23, for example, will depend on the length of the wearers forearm, and the size of the pliable body 11 will depend on the size of the wearer's hand.

The present disclosure contemplates article 10 including removable padding 18 and/or removable forearm guard 23. The removability of the padding 18 and/or forearm guards 23 can be achieved through various means including, but not limited to, a closure (not shown) along casing. The present disclosure further contemplates any number of padding 18 and/or forearm guards 23 that are usable with the article 10 so that a kit is provided. The article 10 can thus be customized for the particular needs of the wearer including particular sizes, particular activities and particular levels of protection by providing various interchangeable sizes and/or shapes and/or materials for the padding 18 and forearm guards 23.

While several arrangements of the invention have been described, it will be understood that it is capable of still further modifications and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention or the limits of the appended claims.

I claim:

1. A protective article comprising:
a body having a finger portion and a forearm portion, wherein said forearm portion extends from a wearers first finger knuckles to a location past a wearers wrist joint;
a forearm guard being substantially rigid and extending from at least said forearm portion to a location past the wearers wrist joint; and
a wrist wrap having at least one fastener and operable to secure said forearm guard adjacent to a dorsal side of the wearers wrist joint to support the wearer's wrist joint.

2. The protective article of claim 1, further comprising:
a padding extending over at least a portion of a dorsal side of said body, the padding having a knuckle pad region and a forearm pad region, wherein said forearm pad region extends from beyond the wearers first finger knuckles to a location past the wearers wrist joint; and
a flexible joint at the juncture between said knuckle pad region and said forearm pad region, wherein said forearm guard covers at least a portion of said forearm pad region.

3. The protective article of claim 1, wherein said forearm guard extends from at least said flexible joint to said location past a wearers wrist joint.

4. The protective article of claim 1, wherein said wrist wrap is attached to said body at a first end and having said at least one fastener at a second end.

5. The protective article of claim 4, wherein said first end of said wrist wrap is removably attached to said body.

6. The protective article of claim 1, wherein said wrist wrap further comprises an elastic material.

7. The protective article of claim 2, wherein said knuckle pad region and said forearm pad region are separate pieces of padding.

8. The protective article of claim 2, wherein a thickness of said knuckle pad region is greater than a thickness of said forearm pad region.

9. The protective article of claim 2, wherein said forearm guard and said forearm pad region are shaped to generally conform to a dorsal contour of a wearers hand, wrist and forearm.

10. The protective article of claim 1, wherein said body further comprises a fingerless glove, said finger portion extending only a part way along a wearers fingers.

11. The protective article of claim 1, wherein a palmar side of said body comprises a closed finger portion, an open palm portion and a closed wrist portion.

12. The protective article of claim 11, wherein said wrist portion comprises an elastic band attached at each lateral edge of said body.

13. The protective article of claim 1, wherein said forearm guard comprises a plurality of adjacent forearm guard strips, each one of said strips extending from said flexible joint to a location past the wearer's wrist joint.

14. A protective article comprising:
a body having a finger portion and a forearm portion, wherein said forearm portion extends from a wearers first finger knuckles to a location past a wearer's wrist joint;
a padding extending over at least a portion of a dorsal side of said body, the padding having a knuckle pad region and a forearm pad region, wherein said forearm pad region extends from beyond the wearers first finger knuckles to a location past the wearer's wrist joint;
a casing surrounding at least a portion of said padding;
a flexible joint at the juncture between said knuckle pad region and said forearm pad region;
a forearm guard being substantially rigid and extending from at least said flexible joint to a location past a wearer's wrist joint, wherein said forearm guard covers at least a portion of said forearm pad region; and
a wrist wrap having at least one fastener and operable to secure said forearm guard adjacent to a dorsal side of the wearer's wrist joint to support the wearers wrist joint.

15. The protective article of claim 14, wherein said knuckle pad region and said forearm pad region are separate pieces of padding.

16. The protective article of claim 14, wherein a thickness of said knuckle pad region is greater than a thickness of said forearm pad region.

17. The protective article of claim 14, wherein said forearm guard and said forearm pad region are shaped to generally conform to a dorsal contour of a wearer's hand, wrist or forearm.

18. A method of supporting a wearer's wrist joint comprising:
positioning a substantially rigid forearm guard on a dorsal side of a wearers hand from at least in proximity to a wearers knuckles to a location past a wearers wrist joint;
holding said substantially rigid forearm guard in position via a glove body; and
securing said substantially rigid forearm guard against said dorsal side of the wearers hand via a wrist wrap to support the wearers wrist joint.

19. The method of claim 18, further comprising:
positioning padding on the dorsal side of a wearers hand; and
holding said padding in position via a casing surrounding said padding and operably connected to said glove body, wherein said glove body has a finger portion and a forearm portion, and wherein said forearm portion extends from a wearers first finger knuckles to a location past the wearers wrist joint.

20. The method of claim 19, wherein at least one of said substantially rigid forearm guard or said padding is removable from said glove body.
